(19) United States
(12) Patent Application Publication
(10) Pub. No.: US 2011/0112331 A1
(43) Pub. Date: May 12, 2011

(54) Method for phase separation of polytrimethylene ether glycol in salt solution

(75) Inventors: Arthur William Etchells, Philadelphia, PA (US); Frederic Godbille, Kingston (CA); Hua Liang, Kingston (CA); Hari Babu Sunkara, Hockessin, DE (US)

(73) Assignee: E.I. DU PONT DE NEMOURS AND COMPANY, Wilmington, DE (US)

(21) Appl. No.: 12/614,516

(22) Filed: Nov. 9, 2009

(51) Int. Cl. C07C 43/10

(52) U.S. Cl. 568/619

(57) ABSTRACT

Processes are provided for manufacturing polytrimethylene ether glycol, in which water is added to the product polymer to facilitate phase separation.

### FIELD OF THE INVENTION

The present invention relates to an improved process of manufacture of polytrimethylene ether glycol.

### BACKGROUND

Methods for preparation of polytrimethylene ether glycol (hereinafter also referred to as “PO3G”) include acid catalyzed polycondensation of 1,3-propanediol (hereinafter also referred to as “PDO”). For example, U.S. Pat. Nos. 6,720,459 and 6,977,291 disclose processes for preparation of polytrimethylene ether glycol from 1,3-propanediol using a polycondensation catalyst, preferably an acid catalyst. Previous methods of purifying polytrimethylene ether glycol produced by acid-catalyzed polycondensation reactions have included a hydrolysis step wherein water is added to the crude polymer to hydrolyze acid esters found in the aqueous-organic mixture. Subsequent steps include separating the water phase and the organic phase by allowing the mixture to stand until phase separation is considered completed. Approaches to accelerating phase separation have been reported (for example in U.S. Pat. Nos. 7,388,115 and 7,161,045), however use of these approaches generally necessitate additional steps or reagents (addition of organic solvent, for example, as in U.S. Pat. No. 7,161,045), and leave room for improvement for expedient phase separation of high molecular weight polymer, particularly in a manner which retains high quality polymer product.

Commonly owned U.S. Pat. No. 7,388,115 discloses a procedure for making polytrimethylene ether glycol via an acid catalyzed polymerization process comprising hydrolysis of the acid esters formed during the acid catalyzed polymerization, addition of one or more water-soluble, inorganic compounds to form an aqueous-organic mixture, separating the aqueous phase and the organic phase, and removing residual water from the organic phase. Additional steps are also disclosed. One such step is optionally adding base to the separated organic phase to neutralize residual acid polycondensation catalyst by forming salts. Also disclosed is using organic solvents miscible with polytrimethylene ether glycol to promote phase separation as described in U.S. Pat. No. 7,161,045.

Despite the efforts and decreases in phase separation time described in the art, there remains a need to minimize time and processing steps necessary to purify polytrimethylene ether glycol produced via acid-catalyzed polycondensation.

### SUMMARY OF THE INVENTION

One aspect of the present invention is a process for manufacturing polytrimethylene ether glycol consisting essentially of:
a. polycondensing reactants comprising at least one diol selected from the group consisting of 1,3-propanediol, 1,3-propanediol dimer, and 1,3-propanediol trimer and mixtures thereof in the presence of an acid polycondensation catalyst to form polytrimethylene ether glycol, wherein acid esters are formed during the polycondensation;
b. adding water to the polytrimethylene ether glycol such that the polytrimethylene ether glycol to water ratio is about 1:1 or lower and hydrolyzing the acid esters to form a hydrolyzed mixture containing the polytrimethylene ether glycol and the hydrolyzed acid esters;
c. adding one or more water-soluble, inorganic compounds to the hydrolyzed mixture to form an aqueous-organic mixture comprising (i) an organic phase containing polytrimethylene ether glycol, (ii) a coalescence band, and (iii) an aqueous phase
d. allowing the phases to separate at least until the coalescence band disappears and a degree of separation is achieved;
e. decanting the aqueous phase from the organic phase containing polytrimethylene ether glycol; and
f. drying and filtering the organic phase containing polytrimethylene ether glycol.

### BRIEF DESCRIPTION OF THE DRAWINGS

FIG. 1. Schematic of setup to test liquid-liquid phase separation rate.

FIG. 2. A) Height of the coalescence band in the column over time at 80° C. for polymer of Mw 650 in 4% sodium carbonate solution and 2:1 polymer to water ratio.

B) Height of the aqueous/coalescence interface (A&B) and coalescence/polymer interface (B&C) in the column over time at 80° C. for polymer of Mw 650 in 4% sodium carbonate solution and 2:1 polymer to water ratio.

FIG. 3. A) Photographic changes in aqueous phase (bottom), coalescence band (middle) and polymer phase (top) at 7, 16, 20 and 28 minutes of separation time at 80° C. for Mw 650 in 4% sodium carbonate and 2:1 polymer to water ratio.

B) Schematic example of phases: aqueous phase (“phase A”, bottom), coalescence band (“phase B”, middle), and polymer phase (“phase C”, top).

### DETAILED DESCRIPTION

Provided herein is an improved process for production and purification of polytrimethylene ether glycol manufactured using a sulfuric acid catalyst.

The starting material for the process is a reactant comprising at least one of 1,3-propanediol, 1,3-propanediol dimer and 1,3-propanediol trimer, or mixtures thereof. The 1,3-propanediol reactant can be obtained by any of the various chemical routes or by biochemical transformation routes.

A particularly preferred source of 1,3-propanediol is via a fermentation process using a renewable biological source. As an illustrative example of a starting material from a renewable source, biochemical routes to 1,3-propanediol have been described that utilize feedstocks produced from biological and renewable resources such as corn feed stock. For example, bacterial strains able to convert glycerol into 1,3-propanediol are found in the species Klebsiella, Citrobacter, Clostridium, and Lactobacillus. The technique is disclosed in several publications, including U.S. Pat. No. 5,633,362, U.S. Pat. No. 5,686,276 and U.S. Pat. No. 5,821,092. U.S. Pat. No. 5,821,092 discloses, inter alia, a process for the biological production of 1,3-propanediol from glycerol using recombinant organisms. The process incorporates E. coli bacteria, transformed with a heterologous pdu diol dehydratase gene, having specificity for 1,2-propanediol. The transformed E. coli is grown in the presence of glycerol as a carbon source and 1,3-propanediol is isolated from the growth media. Since both bacteria and yeasts can convert glucose (e.g., corn sugar) or other carbohydrates to glycerol, the processes disclosed in these publications provide a rapid, inexpensive and environmentally responsible source of 1,3-propanediol monomer.

The biologically-derived 1,3-propanediol, such as produced by the processes described and referenced above, contains carbon from the atmospheric carbon dioxide incorporated by plants, which compose the feedstock for the production of the 1,3-propanediol. In this way, the biologically-derived 1,3-propanediol contains only renewable carbon, and not fossil fuel-based or petroleum-based carbon. The polytrimethylene terephthalate based thereon utilizing the biologically-derived 1,3-propanediol, therefore, has less impact on the environment as the 1,3-propanediol used does not deplete diminishing fossil fuels and, upon degradation, releases carbon back to the atmosphere for use by plants once again. Thus, the compositions can be characterized as more natural and having less environmental impact than similar compositions comprising petroleum based diols. Preferably the 1,3-propanediol used as a reactant or as a component of the reactant in making the polymer polytrimethylene ether glycol will have a purity of greater than about 99%, and more preferably greater than about 99.9%, by weight as determined by gas chromatographic analysis. Particularly preferred are the purified 1,3-propanediols as disclosed in U.S. Pat. No. 7,038,092, U.S. Pat. No. 7,098,368, U.S. Pat. No. 7,084,311 and US20050069997A1.

Although any of 1,3-propanediol, and dimers or trimers of 1,3-propanediol can be used as a reactant, it is preferred that the reactant comprise about 90% or more by weight of 1,3-propanediol. More preferably the reactant will comprise 99% or more by weight of 1,3-propanediol.

Any acid catalyst suitable for acid catalyzed polycondensation of 1,3-propanediol may be used in present process. Preferred acid polycondensation catalysts are described in U.S. Published Patent Application No. 2002/0007043 A1 and in U.S. Pat. No. 6,720,459. They are preferably selected from the group consisting of Lewis acids, Bronsted acids, super acids, and mixtures thereof, and they include both homogeneous and heterogeneous catalysts. More preferably the catalysts are selected from the group consisting of inorganic acids, organic sulfonic acids, heteropolyacids and metal salts. Most preferably the catalyst is a homogeneous catalyst, preferably selected from the group consisting of sulfuric acid, hydriodic acid, fluorosulfonic acid, phosphorous acid, p-toluenesulfonic acid, benzenesulfonic acid, methanesulfonic acid, phosphotungstic acid, trifluoromethanesulfonic acid, phosphomolybdic acid, 1,1,2,2-tetrafluoro-ethanesulfonic acid, 1,1,1,2,3,3-hexafluoropropanesulfonic acid, bismuth triflate, yttrium triflate, ytterbium triflate, neodymium triflate, lanthanum triflate, scandium triflate, and zirconium triflate. The catalyst can also be a heterogeneous catalyst, preferably selected from the group consisting of zeolites, fluorinated alumina, acid-treated alumina, heteropolyacids and heteropolyacids supported on zirconia, titania, alumina and/or silica. The most preferred catalyst is sulfuric acid.

The polymerization process can be batch, semi-continuous, continuous, etc. A preferred batch process is described in US 2002/0007043 A1. In this embodiment the polytrimethylene-ether glycol is prepared by a process comprising the steps of: (a) providing (1) reactant, and (2) acid polycondensation catalyst; and (b) polycondensing the reactants to form a polytrimethylene ether glycol. The reaction is conducted at an elevated temperature of at least about 150 degrees C., more preferably at least about 160 degrees C., The reaction is preferably conducted either at atmospheric pressure in the presence of inert gas or at reduced pressure (i.e., less than 1 atmosphere), preferably less than about 500 mm Hg in an inert atmosphere and extremely low pressures can be used (e.g., as low as about 1 mm Hg). Suitable inert gases are known to those of skill in the art, and nitrogen is preferred.

A preferred continuous process for preparation of the polytrimethylene ether glycols is described in U.S. Pat. No. 6,720,459. Thus, in one embodiment the polytrimethylene ether glycol is prepared by a continuous process comprising: (a) continuously providing (i) reactant, and (ii) polycondensation catalyst; and (b) continuously polycondensing the reactant to form polytrimethylene ether glycol.

A next step comprises hydrolyzing the acid esters formed during the polycondensation that are in the aqueous-organic mixture. When a homogeneous acid catalyst, particularly sulfuric acid, is used for production of polytrimethylene ether glycol, a substantial amount of acid ester is formed from the catalyst. In the case of sulfuric acid, a substantial portion of the acid is converted to the ester, alkyl hydrogen sulfate. It is important to remove these acid esters because they act as emulsifying agents during the water washing used to remove catalyst and therefore cause the washing process to be difficult and time consuming. Moreover, the hydrolysis step is also important in order to obtain polymer with the high dihydroxy functionality required to use the polymer as a reactive intermediate.

The hydrolysis step is carried out by adding water to the polymer and by heating the aqueous-organic mixture to at a temperature range from about 80 to about 110 degree C., or at about 90 to about 110 degrees C. (or at about 90 degree C. when carried out at atmospheric pressure) for a sufficient time with sufficient agitation. Hydrolysis is preferably carried out at less than about 90 degrees C. for about 4 to 6 hours. The hydrolysis step is preferably conducted at atmospheric or slightly above atmospheric pressure, preferably at (about 700 mmHg to about 1600 mmHg). Higher pressures can be used, but complicate processes and increase equipment cost, therefore are not preferred. The hydrolysis step is carried out under inert gas atmosphere.

When the hydrolysis step is incorporated into the process, it has been observed that often the phase separation between the aqueous and polytrimethylene ether glycol phases can take a substantial amount of time, as long as several hours, until the phases are separated sufficiently to proceed further. It has been observed that generally, the higher the polymer molecular weight is, the longer it takes to separate two phases. It has also been found that utilizing an excess of water during or following the hydrolysis step speeds the phase separation without necessitating further purification steps to achieve suitable polytrimethylene ether glycol.

While not wishing to be bound by theory, it is believed that in some cases the processes disclosed herein can cause a phase inversion wherein the addition of water causes the aqueous phase to become the continuous phase. Further, because water is a reagent added during the process to hydrolyze the acid esters, no additional hydrolysis reagents are used, and reagent-specific purification and handling steps need not be added to the process.

The amount of water is preferably at least about 100 to about 200 weight percent of polytrimethylene ether glycol, or, expressed as a ratio of polymer to water about 1:1 to about 1:2. It is preferred that the ratio of polymer to water is less than or equal to about 1:1. That is, it is preferred that there is at least one part water per one part of polytrimethylene ether glycol. Larger excesses of water can also be used, and the skilled artisan will appreciate the practicalities and trade-offs, including the size of the vessel to which the water will be added.

In some embodiments, the ratio of polymer to water is greater than or equal to about 1:2. In such embodiments, there are about two parts or less of water per one part of polytrimethylene ether glycol.

The hydrolysis and the addition of an excess of water for phase separation can be carried out concurrently or may be separated in time. For example, water can be added to the reaction mixture in an amount sufficient for hydrolysis but less than the amount necessary for phase separation. The mixture can then be subjected to conditions appropriate for hydrolysis prior to bringing the total water in the reaction mixture to an amount of about 100 to about 200 weight percent of the polymer.

To the aqueous-organic mixture after hydrolysis is added one or more water-soluble, inorganic compounds to form an aqueous-organic mixture comprising (i) an organic phase containing polytrimethylene ether glycol and (ii) an aqueous phase.

The water-soluble, inorganic compounds are preferably inorganic salts and/or inorganic bases. Preferred salts are those comprising a cation selected from the group consisting of ammonium ion, Group IA metal cations, Group IIA metal cations and Group IIIA metal cations, and an anion selected from the group consisting of fluoride, chloride, bromide, iodide, carbonate, bicarbonate, sulfate, bisulfate, phosphate, hydrogen phosphate, and dihydrogen phosphate (preferably chloride, carbonate and bicarbonate). Group IA cations are lithium, sodium, potassium, rubidium, cesium and francium cations (preferably lithium, sodium and potassium); Group IIA cations are beryllium, magnesium, calcium, strontium, barium and radium (preferably magnesium and calcium); and Group IIIA cations are aluminum, gallium, indium and thallium cations. More preferred salts are ammonium chloride, lithium chloride, sodium chloride, potassium chloride, magnesium chloride, calcium chloride, sodium carbonate and sodium bicarbonate. The most preferred salt is sodium carbonate or soda ash.

Suitable inorganic bases include ammonium hydroxide and water-soluble hydroxides derived from any of the above-mentioned Group IA, IIA and IIIA metal cations. The most preferred water-soluble inorganic bases are sodium hydroxide and potassium hydroxide. The amount of water-soluble, inorganic compound used can vary, but is preferably the amount effective in promoting the rapid separation of the water and inorganic phases. The preferred amount for this purpose is from about 1 to about 20 wt %, more preferred is an amount from about 1 to about 10 wt %, and still more preferably an amount from about 2 to about 8 wt %, based on the weight of the water added to the polytrimethylene ether glycol in the hydrolysis step.

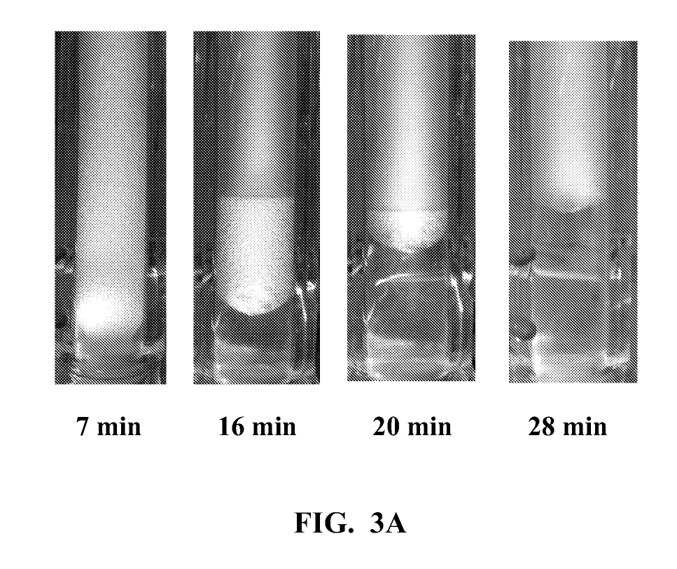

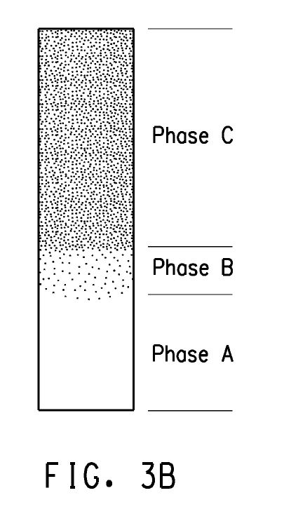

The next step comprises separating the aqueous phase and the organic phase. Separation is preferably carried out by allowing the aqueous phase and the organic phase to separate and settle so that the aqueous phase can be removed. The reaction mixture is allowed to stand, without agitation, until settling and the desired phase separation has occurred. A preferred indication of completed phase separation is the disappearance of the coalescence band (see, for example, FIG. 3). The time required for separation and settling will be less than that required for a process that is the same except for the use of less water. In some instances, use of the methods described herein allow for separation of reaction mixtures where separation was not previously observed.

The separation can be carried out at a range of temperatures, preferably at a temperature of about 50 to about 100 degrees C., and more preferably at a temperature of about 80 to about 90 degrees C.

The “degree of separation” as used herein is the weight of the aqueous phase as a percentage of the total weight of the pre-separated aqueous-organic mixture. Prior to completion of phase separation, the aqueous-organic mixture comprises (i) an organic phase containing polytrimethylene ether glycol and residual acid polycondensation catalyst from the polycondensing, (ii) a coalescence band, and (iii) an aqueous phase. The degree of separation is preferably determined after the disappearance of the coalescence band.

The degree of separation may be greater than about 60%, but more preferred is a degree of separation greater than about 80% and in some embodiments greater than about 90%. Typically the higher the degree of separation, the less load the down stream process will encounter in commercial production.

The processes disclosed herein provide shorter periods for phase separation after hydrolysis than some previously known methods.

Preferably the time required for phase separation according to the processes disclosed herein is less than about 1 hour. More preferably this time period is from less than about 30 minutes, and most preferably about 5 minutes or less. In general, the shorter the time for phase separation, the shorter the production cycle. In some embodiments, the coalescence band disappears in less than about 100 minutes or less than 60 minutes or less than 10 minutes. The droplet settling rate of the dispersed phase can be calculated using Stokes' Law (see Example 8). It is preferred that the droplet settling rate is greater than 0.

U.S. patent application Ser. No. 11/204,713 and Ser. No. 11/204,731 (filed disclose processes for preparing polytrimethylene ether glycol by acid polycondensation wherein the phase separation after hydrolysis is promoted by addition of organic solvent miscible with polytrimethylene ether glycol in the case of U.S. patent application Ser. No. 11/204,713 (filed Aug. 16, 2005), and miscible with water in the case of U.S. patent application Ser. No. 11/204,731 (filed Aug. 16, 2005). It is preferred that the solvents disclosed in these two applications are not used conjunction with the water excess and water-soluble inorganic compounds disclosed herein to promote phase separation. The hydrolysis step of the processes disclosed herein is preferably carried out in the absence of an organic solvent, thus simplifying the process and eliminating the need for procurement and handling of additional reagents yet still producing desirable increases in speed of phase separation.

Once phase separation has occurred, the aqueous phase and the organic phase can be split, preferably by decantation or draining. It is advantageous to drain off the aqueous phase and retain the organic phase in the reactor for subsequent processing.

Optionally, following the hydrolysis and phase separation steps, a base, preferably a substantially water-soluble base can be added to neutralize any remaining acid. During this step residual acid polycondensation catalyst is converted into its corresponding salts.

Preferably, the base is selected from the group consisting of alkaline earth metal hydroxides and alkaline earth metal oxides. More preferably, the base is selected from the group consisting of calcium hydroxide, calcium oxide, magnesium hydroxide, magnesium oxide, barium oxide and barium hydroxide. Mixtures can be used. A particularly preferred base is soda ash. The base can be added as a dry solid, or preferably as an aqueous slurry. The amount of soluble base utilized in the neutralization step is preferably at least enough to neutralize all of the acid polycondensation catalyst. More preferably a stoichiometric excess of from about 0.1 wt % to about 10 wt % is utilized. The neutralization is preferably carried out at 50 to 90.degree. C. for a period of from 0.1 to 3 hours under nitrogen atmosphere.

Next, in a drying step, any residual hydrolysis water is preferably removed from the organic phase by vacuum stripping (e.g., distillation at low pressure), generally with heating, which will also remove organic solvent if present and, if desired, unreacted monomeric materials. Other techniques can be used, such as distillation at about atmospheric pressure.

When base is added such that residual acid catalyst salts are formed, the organic phase is separated into (i) a liquid phase comprising the polytrimethylene ether glycol, and (ii) a solid phase comprising the salts of the residual acid polycondensation catalyst and unreacted base. Typically, this is carried out by filtration (preferably with use of filter-aid material, for example as disclosed in U.S. Pat. No. 2005/028302A1, or centrifugation, to remove the base and the acid/base reaction products. This step can be referred to as the “filtration step”. Centrifugation and filtration methods are generally well known in the art. For example, filtration under gravity, centrifugal filtration, or pressure filtration can be used. Filter presses, candle filters, pressure leaf filters or conventional filter papers are also be used for the filtration, which can be carried out batch wise or continuously. Filtration in the presence of a filter-aid is preferred at a temperature range from 50 to 100.degree. C. at a pressure range from 1 to 5 bars.

Even if base is not added, a “filtration step” using purification techniques like centrifugation and filtration may still be desirable for refining the product.

The remaining polytrimethylene ether glycol will preferably have molecular weight (Mw) of from about 250 to about 5000. Molecular weight of 500 to 3000 is preferred for many applications. The polytrimethylene ether glycol will preferably have a low residual sulfur content. Residual sulfur content can be measured using methods known in the art, such as by X-ray Fluorescence (XRF). Final residual sulfur content is a quality characteristic of the polymer product which may affect the selection of the product for a particular application. In some embodiments, the residual sulfur content is less than 15 ppm. In some embodiments, the residual sulfur content is less than 10 ppm, and in some embodiments, it is less than 5 ppm. Residual sulfur content may be determined by X-ray Fluorescence and is a quality characteristic of the polymer product which may affect the selection of the product for a particular application.

Generally, the higher the molecular weight of the polytrimethylene ether glycol, the longer the phase separation time, thus, the processes disclosed herein become more advantageous as molecular weight increases. Using previous processes, particularly for high molecular weight polymers such as those having molecular weights of about 1400 or greater, the phase separation step took hours or even days, and thus may have been omitted or abbreviated. Because less hydrolysis water was decanted, subsequent drying time and energy consumed in drying was increased.

### EXAMPLES

### Comparative Example A

### Polymer of Mw 650, 4% Sodium Carbonate Solution, 2:1 Polymer to Water Ratio

Polymerization: 12 kg of bio-based 1,3-propanediol monomer, 110.8 g sulfuric acid and 55.9 g sodium carbonate solution (10%) were added to a 20 L glass reactor equipped with a condenser and an agitator. The mixture was mixed with agitation speed of 150 rpm and purged with N₂ of rate 10 L/min for 30 minutes before it was heated to 166° C. The time when heating is turned on was set as the reaction starting point. Polymerization proceeded at 166° C. The reaction volatiles were condensed in the condenser and collected in a receiver. Crude polymer product was accumulated in the reactor. At an estimated reaction time, the crude polymer samples were taken periodically for molecular weight analysis. Viscosity of the crude polymer samples were measured on a Brookfield Viscometer (DV-11+ Pro; Brookfield Engineering Laboratories, MA, USA) and correlated to the number average molecular weight of the crude polymer via a previously established calibration curve. For Mw 650, after approximately 12.8 hours of heating, the viscosity of the crude polymer sample reached 297 cP at 25° C., which corresponds to a molecular weight of 608. After reaching the desired crude molecular weight, N₂ flow was reduced to 1 L/min and heating and agitation were reduced The amount of the crude polymer in the reactor was calculated to be about 8 kg from the mass balance of starting ingredients, condensate, and collections in bubbler. Final molecular weight of the crude polymer was also later confirmed by nuclear magnetic resonance (NMR) method (Bruker, AVANCE-500).

Hydrolysis: 4 liters of distilled water were pumped into the crude polymer in reactor, using a Peristaltic Masterflex® pump (Barnant Co., Barrington, Ill., USA). After water addition was completed, the N₂ flow was set to 1 L/min and agitation speed to 150 rpm. The liquid temperature was monitored and controlled until it reached the hydrolysis temperature of 95° C., marking the start time for 6 hour hydrolysis.

Neutralization: 180 g of soda ash was dissolved in 480 g lukewarm distilled water to make sodium carbonate solution. After 6 hours of hydrolysis, the heating and N₂ flow were turned off. Agitation was reduced to about 50 rpm, and sodium carbonate solution was slowly added into the reactor, using a Peristaltic Masterflex® pump. Mixing continued for at least 60 minutes after addition of sodium carbonate solution was completed. Samples of the mixture (about 150 mL) were taken for phase separation experiments.

### Comparative Example B

### Polymer of Mw 1400, 4% Sodium Carbonate Solution, 2:1 Polymer to Water Ratio

The equipment, polymerization, hydrolysis and neutralization procedures were the same as in Comparative Example A except that for Mw1400 the polymerization time at 166° C. was 20.1 hours.

### Comparative Example C

### Polymer of Mw 2400, 4% Sodium Carbonate Solution, 2:1 Polymer to Water Ratio

The equipment, polymerization, hydrolysis and neutralization procedures were the same as in Comparative Example A except that for Mw 2400 the polymerization time at 166° C. was 29.7 hours.

### Example 1

### Polymer of Mw650, 4% Sodium Carbonate Solution, 1:2 Polymer to Water Ratio

The equipment, polymerization and neutralization procedures were the same as in Comparative Example A except that for obtaining 1:2 polymer to water ratio, 8 liters of distilled water was added to 4 kg of crude polymer for hydrolysis.

### Example 2

### Polymer of Mw1400, 4% Sodium Carbonate Solution, 1:2 Polymer to Water Ratio

The equipment, polymerization and neutralization procedures were the same as in Comparative Example B except that for obtaining 1:2 polymer to water ratio, 8 liters of distilled water was added to 4 kg of crude polymer for hydrolysis.

### Example 3

### Polymer of Mw2400, 4% Sodium Carbonate Solution, 1:2 Polymer to Water Ratio

The equipment, polymerization and neutralization procedures were the same as in Comparative Example C except that for obtaining 1:2 polymer to water ratio, 8 liters of distilled water was added to 4 kg of crude polymer for hydrolysis.

### Example 4

### Polymer of Mw650, 4% Sodium Carbonate Solution, 1:1 Polymer to Water Ratio

The equipment, polymerization and neutralization procedures were the same as in Comparative Example A except that for obtaining 1:1 polymer to water ratio, 4 liters of distilled water was added to 4 kg of crude polymer for hydrolysis.

### Example 5

### Polymer Mw1400, 4% Sodium Carbonate Solution, 1:1 Polymer to Water Ratio

The equipment, polymerization and neutralization procedures were the same as in Comparative Example B except that for obtaining 1:1 polymer to water ratio, 4 liters of distilled water was added to 4 kg of crude polymer for hydrolysis.

### Example 6

### Polymer Mw2400, 4% Sodium Carbonate Solution, 1:1 Polymer to Water Ratio

The equipment, polymerization and neutralization procedures were the same as in Comparative Example C except that for obtaining 1:1 polymer to water ratio, 4 liters of distilled water was added to 4 kg of crude polymer for hydrolysis.

### Comparative Example D

### Polymer Mw650, 4% sodium carbonate Solution, 4:1 Polymer to Water Ratio

The equipment, polymerization and neutralization procedures were the same as in Comparative Example A except that for obtaining 4:1 polymer to water ratio, 2 liters of distilled water was added to 8 kg of crude polymer for hydrolysis.

### Comparative Example E

### Polymer Mw650, 8% sodium carbonate Solution, 2:1 Polymer to Water Ratio

The equipment, polymerization, hydrolysis and neutralization procedures were the same as in Comparative Example A except for final 8% sodium carbonate solution, 360 g soda ash was dissolved in 500 gram lukewarm distilled water. This slurry was added to the reactor containing hydrolyzed polymer.

### Example 7

### Determining Liquid-Liquid Phase Separation Rate

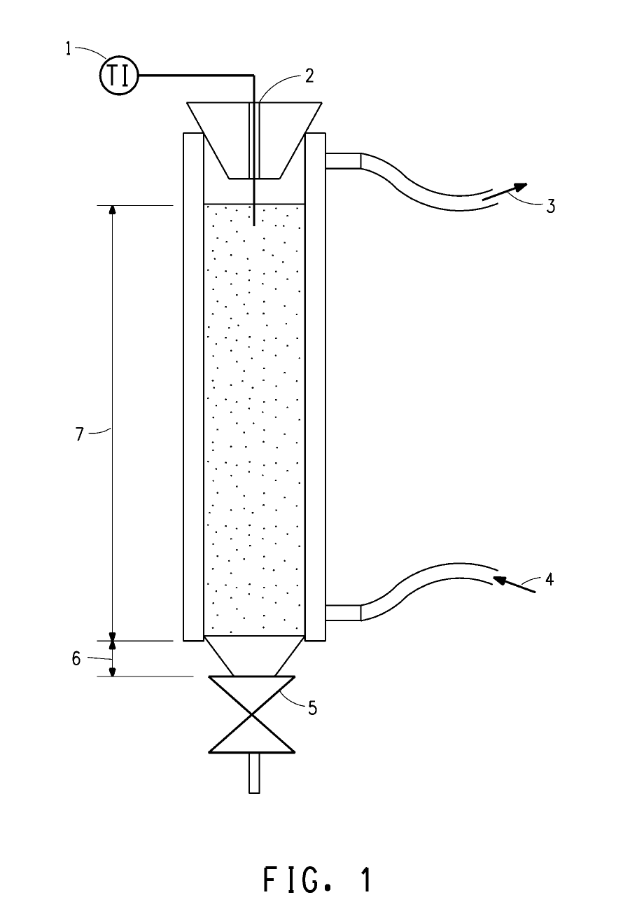

An experimental set up (jacketed glass column) was put up in the fume hood as illustrated in FIG. 1. The jacketed column has a height (7) of about 12 inches or 295 mm, a heel (6) of about 7-8 mL, and has a ¾ inch inside-diameter. The column provides a profile L/D of 15. The liquid temperature in the glass column was controlled to be 60, 80, or 90° C. by a VWR water heating system. The water jacket inflow (4) and outflow (3) are depicted in FIG. 1, and temperature was monitored by a temperature indicator (1). Samples made as described in Examples 1-6 and Comparative Examples A-E were heated close to the test liquid temperature in a hot water bath and were manually vigorously shaken for ˜1 min to ensure the two phases were well mixed. The sample was rapidly poured into the glass column via a funnel (2) at the top of the column. Time zero was marked once the column was filled.

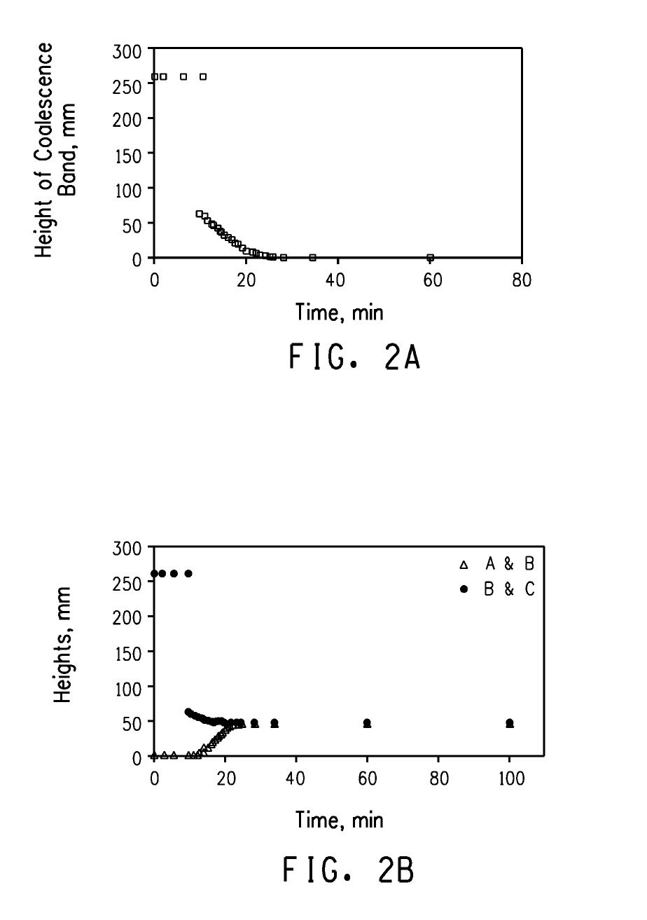

The remaining experimental procedure is described for the sample prepared as described in Comparative Example A, but is the same procedure carried out for all of the samples with results shown in Table 1 (Examples 1-6 and Comparative Examples A-E). For a sample prepared as described in Comparative Example A, within 10 minutes, three regions appeared in the column (see FIGS. 3A and 3B, for example). The three regions were termed aqueous phase (“phase A”, bottom), coalescence, or interphase, band (“phase B”, middle) and polymer phase (“phase C”, top) respectively. The height of each region was measured and observed changing with time. The changes of the heights of the interfaces of the phases are shown in FIG. 2. Pictures of one example experimental sample in the column at 7 min., 16 min., 20 min., and 28 min. are shown in FIG. 3A.

The time of disappearance of the coalescence band is considered to be the separation time. For the example shown in FIG. 3A, the coalescence band disappeared at about 28 minutes. At this time, the top phase was about 63% total volume. The relative change of each phase was continually monitored for a total of about 100 minutes. At the end of 100 minutes, both phases were drained from the column drain valve (5) and weighed to confirm the polymer phase as a percentage of total sample volume, which is expressed in Table 1 as “Final Degree of Separation”. When the separation occurred very quickly, it was difficult to determine which phase was continuous (labeled as “undetermined” in the table).

TABLE 1
Results from Example 7

| | | | | | Time of | | | |
|---|---|---|---|---|---|---|---|---|
| | | | | | disappearance of | | | Calculated rate of |
| Target | Temperature | Polymer/ | Salt Conc, | Continuous | interphase | Final Degree of | | settling by applying |
| Mwt | (° C.) | water ratio | wt % | Phase | (min) | separation | Note | Stokes' Law, cm/s |
| ′650 | 60 | 1:1 | 4 | Polymer | 6.2 | 69% | | −0.00012 |
| | | 2:1 | 4 | Polymer | 53.1 | 68% | a | −0.00012 |
| | | 2:1 | 8 | Polymer | 4.3 | 96% | | −0.00060 |
| | | 4:1 | 4 | No separation | N/A | 0% | | −0.00003 |
| | 80 | 1:2 | 4 | Aqueous | 7 | 97% | | 0.02722 |
| | | 1:1 | 4 | Polymer | 2.7 | 69% | | −0.00029 |
| | | 2:1 | 4 | Polymer | 18 | 69% | a | −0.00029 |
| | | 2:1 | 8 | Polymer | 1 | 95% | | −0.00131 |
| | | 4:1 | 4 | No separation | N/A | 0% | | −0.00029 |
| | 90 | 1:2 | 4 | Aqueous | N/A | 96% | | 0.02941 |
| | | 2:1 | 4 | Polymer | 22.5 | 70% | b | −0.00038 |
| 1400 | 60 | 2:1 | 4 | Polymer | 230 | 56% | | −0.00006 |
| | | 2:1 | 8 | Polymer | 103 | 70% | | −0.00022 |
| | 80 | 1:2 | 4 | Aqueous | 4 | 90% | | 0.03663 |
| | | 1:1 | 4 | Undetermined | N/A | 98% | | 0.03633 |
| | | 2:1 | 4 | Polymer | 249 | 90% | | −0.00015 |
| | | 2:1 | 8 | Polymer | 60 | 73% | | −0.00049 |
| | 90 | 1:2 | 4 | Aqueous | N/A | 92% | | 0.04132 |
| | | 1:1 | 4 | Undetermined | 1.3 | 93% | | 0.04035 |
| | | 2:1 | 4 | Polymer | 248.5 | 71% | b | −0.00020 |
| 2400 | 60 | 2:1 | 4 | No separation | N/A | 0% | | −0.00003 |
| | | 2:1 | 8 | No separation | N/A | 0% | | −0.00008 |
| | 80 | 1:2 | 4 | Aqueous | 3 | 84% | | 0.04411 |
| | | 2:1 | 4 | No separation | N/A | 0% | | 0.04817 |
| | | 2:1 | 8 | No separation | N/A | 0% | | −0.00017 |
| | 90 | 1:2 | 4 | Aqueous | N/A | 88% | | 0.04817 |
| | | 1:1 | 4 | No separation | N/A | 0% | | 0.00010 |

Note:
a. - Average of two experiments on two polymer samples
b. - Average of two experiments on the same polymer sample

### Example 8

### Calculation of Droplet Settling Rate

Dispersed phase droplet settling rates in the continuous phase are calculated according to Stokes' Law, which provides a theoretical aspect of phase separation based on physical properties of polymer and aqueous phase. Settling rate can also provide a guideline to the design of decanter. Stokes' Law can be expressed as the formula below,

$$Vs = \frac{{D}_{c} - {D}_{d}}{18 {μ}_{c}} {Gd}^{2}$$

Where Vₛ is the settling rate of a drop in cm/s; D_{c} is the continuous phase density in g/cm³; D_{d} is dispersed phase density in g/cm³; μc is the continuous phase viscosity in cP or 10⁻³ g/(cm*s); G is gravity acceleration (980.665 cm/s²); d is the droplet diameter (in this case, assumed to be 125 micron or 0.0125 cm).

Certain conditions and considerations are noted for applying Stokes'Law in the system: Purified polymer densities at different temperatures were used. Density deviation of neutralized, unfiltered, polymer from purified polymer is unknown. Purified polymer viscosities were used in the calculation when polymer was the continuous phase. Viscosity deviation of neutralized polymer from purified polymer is unknown. Water viscosities were used in the calculation when aqueous phase is the continuous phase. The difference in viscosity between water and Na₂SO₄ solution is negligible. Gravity was assumed constant for this calculation, and drop diameter was assumed constant. (125 micron is an arbitrary chosen minimum value for decanter design as real droplet diameter is difficult to measure accurately.)

A negative settling rate indicates that the polymer phase is the continuous phase and that the dispersed phase is comprised of settling aqueous droplets. A positive settling rate means that the aqueous phase is the continuous phase and that the dispersed phase is comprised of rising polymer droplets. The calculated rate of droplet settling is shown in Table 1.

### Comparative Example F

### Purified Polymer of Mw2400, 4% Sodium Carbonate Solution, 2:1 Polymer to Water Ratio

The equipment, polymerization, hydrolysis and neutralization procedures were the same as in Comparative Example C. Neutralization was followed by 24 hours of phase separation in the same 22 L reactor under nitrogen atmosphere. The aqueous phase and the organic phase were split by draining the aqueous phase at the bottom of the reactor. Next the residual water in the organic phase was removed by vacuum stripping with heating to 90-95° C. for 3 hours. Filtration in the present of filter aid under 10 psi nitrogen pressure was conducted at ˜80° C. to take out the residual acid catalyst salts formed during neutralization.

The purified polytrimethylene ether glycol was tested for its molecular weight by NMR method and residual sulfur content by X-ray Fluorescence (XRF). Final residual sulfur content is a quality characteristic of the polymer product which may affect the selection of the product for a particular application.

### Example 9

### Polymer Mw2400, 4% Sodium Carbonate Solution, 1:2 Polymer to Water Ratio

The equipment, polymerization, neutralization and filtration were similar to Comparative Example F, except that the hydrolysis was conducted under 2:1 water to polymer ratio. Results are shown in Table 2. Results shown in Table 2 indicate that the quality of the polymer product is comparable in both cases.

TABLE 2
Results from Comparative Example F and Example 9

| | Target | | | Phase Separation | MW of purified | S content, |
|---|---|---|---|---|---|---|
| Example | MWt | Polymer:Water | [Na₂CO₃] | time (hrs) | polymer by NMR | ppm |
| Comp Ex F | 2400 | 2:1 | 4 | 24 | 2288 | 10 |
| Ex 9 | 2400 | 1:2 | 4 | 0.67 | 2260 | 11 |

What is claimed is:

1. A process for manufacturing polytrimethylene ether glycol consisting essentially of:
g. polycondensing reactants comprising at least one diol selected from the group consisting of 1,3-propanediol, 1,3-propanediol dimer, and 1,3-propanediol trimer and mixtures thereof in the presence of an acid polycondensation catalyst to form polytrimethylene ether glycol, wherein acid esters are formed during the polycondensation;
h. adding water to the polytrimethylene ether glycol such that the polytrimethylene ether glycol to water ratio is about 1:1 or lower and hydrolyzing the acid esters to form a hydrolyzed mixture containing the polytrimethylene ether glycol and the hydrolyzed acid esters;
i. adding one or more water-soluble, inorganic compounds to the hydrolyzed mixture to form an aqueous-organic mixture comprising (i) an organic phase containing polytrimethylene ether glycol, (ii) a coalescence band, and (iii) an aqueous phase
j. allowing the phases to separate at least until the coalescence band disappears and a degree of separation is achieved;
k. decanting the aqueous phase from the organic phase containing polytrimethylene ether glycol; and
l. drying and filtering the organic phase containing polytrimethylene ether glycol.

2. The process of claim 1 wherein the polytrimethylene ether glycol to water ratio is about 1:2 or greater.

3. The process of claim 1 wherein the aqueous phase is continuous.

4. The process of claim 1 wherein the 1,3-propanediol, 1,3-propanediol dimer, 1,3-propanediol trimer, or mixtures thereof, are bio-derived.

5. The process of claim 1 wherein the acid catalyst is sulfuric acid.

6. The process of claim 1 wherein the water-soluble, inorganic compound is sodium carbonate.

7. The process of claim 1 wherein the coalescence band disappears in less than about 100 minutes.

8. The process of claim 1 wherein the coalescence band disappears in less than about 60 minutes.

9. The process of claim 1 wherein the coalescence band disappears in less than about 10 minutes.

10. The process of claim 1 further comprising determining the degree of separation.

11. The process of claim 10 wherein the degree of separation is greater than about 70%.

12. The process of claim 10 wherein the degree of separation is greater than about 90%.

13. The process of claim 1 wherein the polytrimethylene ether glycol has a molecular weight of at least about 1400.

14. The process of claim 1 wherein the polytrimethylene ether glycol has a molecular weight of at least about 2400.

15. The process of claim 1 wherein the polytrimethylene ether glycol contains residual sulfur after drying and filtering the organic phase and the process further comprises determining the residual sulfur content of the polytrimethylene ether glycol.

16. The process of claim 15 wherein the residual sulfur content of the polytrimethylene ether glycol is less than about 15 ppm.
